(19) United States
(12) Patent Application Publication
(10) Pub. No.: US 2004/0127566 A1
(43) Pub. Date: Jul. 1, 2004

(54) Retinoid hepatitis therapy

(76) Inventor: Anthony H William, Medford, MA (US)

Correspondence Address: Pennie & Edmonds, 1155 Avenue of the Americas, New York, NY, 10036-2711, US

(21) Appl. No.: 10/467,096

(22) PCT Filed: Jan. 31, 2002
(86) PCT No.: PCT/US02/02996

(51) Int. Cl. A61K 31/203

(52) U.S. Cl. 514/559

(57) ABSTRACT

This invention is drawn to a method of treating hepatitis comprising administering to a subject in need of such treatment a therapeutically effective amount of retinoid such as all-trans retinoic acid. In particular embodiments, the form of hepatitis is Hepatitis A, B, C, D, E and G and the treatment is with liposomal all-trans retinoic acid.

### FIELD OF THE INVENTION

This invention is drawn to a method of treating hepatitis comprising administering to a subject in need of such treatment a therapeutically effective amount of retinoid such as all-trans retinoic acid. In particular embodiments, the form of hepatitis is viral hepatitis caused by infection with A, B, C, D, E, and G and the treatment is with liposomal all-trans retinoic acid.

### BACKGROUND OF THE INVENTION

Hepatitis is an inflammatory liver disease. It is associated with loss of appetite, dark urine, fatigue, and, occasionally, fever. In particular instances the liver may become enlarged and jaundice may be present. Both chronic and acute hepatitis is known. By definition, the acute form subsides, generally after about eight weeks. This form rarely results in liver failure. Chronic viral hepatitis patients often remain infectious. The chronic viral hepatitis group is at risk of lasting liver disease, such as cirrhosis or hepatocellular carcinoma.

There are a number of causes and types of hepatitis, but hepatitis of viral etiology is most common. Currently identified strains of viral hepatitis are A, B, C, D, E, and G. Note that the names have changed with increased knowledge and more sophisticated tools. Type A was previously termed infectious hepatitis. Type B was previously termed serum hepatitis, and Type C was non-A, non-B hepatitis (with some exceptions). Type D was delta hepatitis. Additional hepatitis viruses are regularly being identified and isolated. Other viruses have been known to exhibit hepatitis as a secondary effect. These viruses include Cytomegalovirus, Epstein-Barr virus, as well as Yellow Fever. Hepatitis is also a secondary effect of certain parasites and bacteria infections. Non-viral hepatitis is also associated with autoimmune diseases, Wilson's disease, hemochromatosis, and those diseases of toxic origin such as drug, chemical, and alcoholic induced liver disease.

Particular attention is drawn to Hepatitis B and C. Hepatitis C was not specifically identified by virus until 1988. Before that it was within the class termed non-A, non-B hepatitis, and, likely, comprising the vast majority of non-A non-B cases. Without being bound by any particular theory, it is believed that an RNA virus of the type described as “Flavivirus” is the cause of Hepatitis C. This RNA virus has been described as a 40-50 nanometer linear single-strand RNA (ribonucleic acid) virus with a lipid envelope. In native state, the lipid envelope is encased with glycoprotein polymers termed “spikes.” Without being bound by any particular theory, it is further believed that Hepatitis B is caused by a double stranded DNA virus of the type known as Hepadnavirus. It is a 42 nm particle containing ds DNA in a core associated with the polymerase, surrounded by a capsule consisting of surface antigen.

Hitherto, there have been no satisfactory treatments for chronic viral hepatitis, and particularly so for Hepatitis B or C. Interferon alpha is the mainstay of therapy, together with specific antiviral compounds, such as ribavirin and lamivudine. Unfortunately, response rates are rarely greater than 50%. Prevention methodology has been suggested for hepatitis B, in the form of a vaccine. Hepatitis C vaccines have not proven effective. However, the prevalence of both these chronic infections is in excess of 2 billion people.

Note is made of the human enteroviruses. These are known to cause numerous illnesses. Taxonomically, enterovirus is classified as a genus of the family Picornaviridae. This genus is generally divided into five major groups. These are (i) polioviruses, (ii) group A coxsackieviruses, (iii) group B coxsackiviruses, (iv) echoviruses and (v) “newer” enteroviruses. Some recent investigators exclude hepatitis A virus from the genus of enterovirus. Enteroviruses are characterized by a high degree of genetic diversity at the VP1 locus. Particular reference is made to Echovirus 3, 4, 6, 7, 9 and 11, as well as Coxsackie A9, B2, B3, B5, and B9.

Note is also made of chronic diseases. These are illnesses that are prolonged, do not resolve spontaneously, and are rarely cured completely. Chronic diseases of viral origin such as Epstein-Barr are particularly noted as is hepatitis B and C.

Available treatments for enteroviral disease and chronic disease with a viral component are unsatisfactory.

### SUMMARY OF THE INVENTION

This invention comprises a method of treating hepatitis comprising administering to a subject in need of such treatment a therapeutically effective amount of all-trans retinoic acid, with particular reference to employing a therapeutically effective amount of all-trans retinoic acid (inter alia, liposomal all-trans retinoic acid) of at least about 10 mg at least about every other day. In some embodiments therapeutically effective amounts of liposomal all-trans retinoic acid are variously at least about 10 mg/m², at least about 15 mg/m², at least about 50, at least about 65 mg/m² (particularly at least about 67.5 mg/m²), at least about 150 mg/m² and further including at least about 100 mg at least about every other day. This method is particularly useful in hepatitis which is viral hepatitis including hepatitis A, B, C, D, E, or G.

This invention also comprises a method of treating hepatitis comprising administering to a subject in need of such treatment a therapeutically effective amount of retinoid. In a specific embodiment this includes retinoid in liposomal form, and further in therapeutically effective amount, optionally at least about every other day. Reference is made to amounts of at least about 10 mg/m², at least about 15 mg/m², at least about 50, at least about 65 mg/m² (particularly at least about 67.5 mg/m²), at least about 150 mg/m² and further including at least about 100 mg

This invention yet further comprises a method of treating enteroviral infection in an infected subject comprising administering to said subject a therapeutically effective amount of all-trans retinoic acid. In a specific embodiment this includes retinoid in liposomal form, and further in therapeutically effective amount, optionally at least about every other day. Reference is made to amounts of at least about 10 mg/m², at least about 15 mg/m², at least about 50, at least about 65 mg/m² (particularly at least about 67.5 mg/m²) at least about 150 mg/m² and further including at least about 100 mg, and more. This method contemplates use of liposomal and free all-trans retinoic acid. Specific enteroviral infections treated by the method are from the group consisting of (i) poliovirus, (ii) group A coxsackievirus, (iii) group B coxsackivirus, (iv) echovirus or (v) “newer” enterovirus. Particular reference is made to Echovirus 3, 4, 6, 7, 9 or 11, and Coxsackie A9, B2, B3, B5, or B9.

In an additional embodiment this invention comprises a method of treating chronic viral disease in a subject comprising administering to said subject a therapeutically effective amount of retinoid., with particular reference to retinoid in liposomal form. In particular subjects a therapeutically effective amount of retinoid is at least about 50 mg at least about every other day.

### DETAILED DESCRIPTION OF THE INVENTION

This invention will be better understood with resort to the following definitions:

A. ATRA refers to all-trans retinoic acid, a retinoid. Retinoids in general include trans-retinoic acid and all-trans-retinol. Other retinoids are retinoic acid methyl ester, retinoic acid ethyl ester, phenyl analog of retinoic acid, etretinate, retinol, retinyl acetate, retinaldehyde, all-trans-retinoic acid, and 13-cis-retinoic acid. Non-liposomal retinoids, often suitable for oral administration, are referred to as “free.” Particular reference is made to “free” ATRA. Some researchers have suggested that the “free” or oral form of ATRA is largely bound to serum proteins or other components after uptake and is not “free” in the sense of in solution and unbound. For convenience of terminology, as used herein, “free” is used with reference to the non-liposomal forms, without regard to the eventual binding state in serum or other biological fluids.

B. Liposomal ATRA or retinoid shall be broadly understood to encompass all lipid associated ATRA or retinoid forms. More narrowly defined, “liposomes” are generally spherical structures comprising lipids, fatty acids, lipid bilayer type structures, unilamellar vesicles and amorphous lipid vesicles. Classically, liposomes are completely closed lipid brayer membranes containing an entrapped aqueous volume. Liposomes may be unilamellar vesicles (possessing a single brayer membrane) or multilamellar vesicles (onion-like structures characterized by multiple membrane bilayers, each separated from the next by an aqueous layer). The bilayer is composed of two lipid monolayers having a hydrophobic “tail” region and a hydrophilic “head” region. The structure of the membrane bilayer is such that the hydrophobic (nonpolar) “tails” of the lipid monolayers orient toward the center of the bilayer while the hydrophilic “head” orient towards the aqueous phase.

Liposomes are vesicles composed of one or more concentric phospholipid bilayers and used medically especially to deliver a drug into the body. As used herein, and for convenience, drug:lipid aggregates will be included within the terms liposome and liposomal. By way of example of such nonliposomal lipid bearing forms, reference is made to U.S. Pat. No. 4,610,868 to Fountain, the teachings of which are incorporated herein by reference.

Reference is made to liposomal-ATRA (“L-ATRA”) and retinoids as disclosed in U.S. Pat. No. 5,811,119 “Formulation and Use of Carotenoids in the Treatment of Cancer” the teachings of which are incorporated herein by reference

For convenience, the term “liposomal-ATRA” or “-retinoid” shall extend to high ratio drug:lipid complexes that are not classically liposomes.

C. Hepatitis shall be broadly construed in reference to inflammatory liver disease associated with loss of appetite, dark urine, fatigue, and, occasionally, fever. Association liver enlargement and jaundice are common. Both chronic and acute hepatitis is contemplated. While currently identified strains of viral hepatitis A, B, C, D, E, and G are noted, all known or later discovered strains of viral hepatitis are also contemplated within this invention.

D. Therapeutically effective amount as to a drug dosage shall mean that dosage that provides the specific pharmacological response for which the drug is administered in responding members of a population of subjects in need of such treatment. As to free ATRA, a therapeutically effective amount shall include about 10, about 15, about 50 to about 150 mg/m² and particularly about 65 mg/m², and further about 90 mg/m². In particular embodiments a therapeutically effective amount is about 50 to about 150 mg administered at least about every other day. As to L-ATRA, a therapeutically effective amount shall mean about 10, about 15, about 50 to about 150 mg/m² and particularly about 65 and 67.5 mg/m². In particular embodiments, a therapeutically effective amount of L-ATRA is about 50 to about 150 mg administered at least about every other day. In particular instances, avoidance of toxic response or maintaining an inhibitory concentration requires dosing more or less often. Smaller twice daily doses are contemplated.

Surprisingly, it has now been discovered that, beyond certain recognized anti-cancer activities, retinoids, including ATRA act to reduce the viral load in hepatitis patients, with particular reference to hepatitis B and C. In a particular embodiment, liposomal retinoid such as L-ATRA is effective.

### EXAMPLE 1

### Liposomal ATRA

A 24 year old female was diagnosed with APL and promptly enrolled in an L-ATRA (Atragen®, Aronex Pharmaceuticals, The Woodlands, Tex.) protocol and began L-ATRA treatment. After 6 therapy doses, she developed nausea, vomiting and headache and was diagnosed with Mallory-Weiss Syndrome and upper gastrointestinal bleeding requiring endoscopy, i.v. medications and blood repositions. She continued receiving L-ATRA with no reduction in dose. After the 7th dose, she developed ATRA Syndrome, requiring a 25% dose reduction of study drug. Her ATRA Syndrome resolved and she had received 21 doses of L-ATRA, at which point she achieved first complete cancer remission.

Eighteen days later, she was admitted to initiate the first course of consolidation therapy. During this admission, she presented with flu-like symptoms and mild jaundice. Blood analysis disclosed a severe increase of hepatic enzymes (SPGT and SGOT), hyperbilirubinemia and an increase of alkaline phosphatase. Serological tests confirmed antibodies to hepatitis-C positive, PCR determination of viral hepatitis-C positive.

In compliance with the study protocol, the patient was removed from the study due to of the development of hepatic disease. After removal from the study, she began use of L-ATRA as single agent on a compassionate use basis for up to 9 months. Her viral load on at the outset of this phase of treatment was 427,174 copies/ml (blood). She began compassionate use treatment receiving L-ATRA 3 times per week at 100 mg or 67.5 mg/m² every other day (QOD). About 3 weeks after treatment began, after 12 doses of L-ATRA, her viral load was <1,000 copies/ml. One month subsequent to this point her viral load was <1,000 copies/ml and her blood chemistry was normal as to hepatitis C (hepatic enzymes (SPGT and SGOT), bilirubin, and alkaline phosphatase). Four months after beginning compassionate use of L-ATRA she was still in first complete remission with PCR status negative and doing well.

### EXAMPLE 2

### Free ATRA

A 34 year old female is diagnosed by serological tests as positive for hepatitis-C.

She is promptly treated with free ATRA at about 100 mg or 67.5 mg/m² every other day (QOD). Her viral load on at the outset of treatment is about 300,00 copies/ml (blood). She receives free-ATRA (oral) 3 times per week. About 3 weeks after treatment begins, after 12 doses of ATRA, her viral load is <1,000 copies/ml. One month subsequent to this point her viral load is <11000 copies/ml and her blood chemistry is normal as to hepatitis C (hepatic enzymes (SPGT and SGOT), bilirubin, and alkaline phosphatase). Four months after beginning use of ATRA she is still in first complete remission with PCR status negative.

### EXAMPLE 3

### Free ATRA

A 54 year old male is diagnosed by serological tests as positive for hepatitis-A.

He is promptly treated with free ATRA at about 100 mg or 67.5 mg/m² every other day (QOD). His viral load on at the outset of treatment is about 300,00 copies/ml (blood). He receives free-ATRA (oral) 3 times per week. About 3 weeks after treatment begins, after 12 doses of ATRA, his viral load is <1,000 copies/ml. One month subsequent to this point his viral load is <1,000 copies/ml and his blood chemistry is normal as to hepatitis A (hepatic enzymes (SPGT and SGOT), bilirubin, and alkaline phosphatase). Four months after beginning use of ATRA he is still in complete hepatitis remission with PCR status is negative.

### EXAMPLE 4

### Liposomal ATRA in Hepatitis B

A 7-year-old Hispanic female with newly diagnosed/previously untreated APL started to receive L-ATRA (ATRAGEN®) QOD at 90 mg/m². At baseline, the medical history and serologic tests were consistent with HBV chronic carrier status of the patient (positive HBsAg, postive HBcAg and positive HBeAg) though LFTs (liver function test) were within the normal limits.

After 7 doses of L-ATRA, a grade I increase in liver transaminases was observed which progressed to Grade III (including the LDH level) after 12 doses (3 weeks later). Serologic tests made 5 days later, the time when the patient achieved hematologic complete remission (HCR), still showed positive HbsAg and HbcAg and HbeAg. Biopsy results on 6½ weeks beginning treatment showed Grade II chronic hepatitis and fibrosis.

Because of concerns that cytotoxic chemotherapy would further harm the patient's hepatic condition, the patient was treated with L-ATRA monotherapy as consolidation treatment. This was started on 10½ weeks after first treatment at 67.5 mg/m² TIW. L-ATRA was again withheld on 6½ later after the patient developed progressive moderate exfoliative dermatitis assessed as related to ATRAGEN and was restarted on one week thereafter. The patient's LFT's remained within the normal limits since that time (4 weeks thereafter).

All cited references and their teachings are incorporated herein by reference.

The compositions of this invention possess valuable pharmacological properties. They inhibit virus proliferation with particular reference to enteroviruses and hepatitis associated viruses in human and veterinary medicine. Administration is contemplated to include chronic, acute or intermittent regimens.

The compositions are particularly useful in treating hepatitis A, B, C, D, E, and G.

In addition, the compositions can be used in in vitro methodologies, including treating cell cultures of hepatic tissue to impede viral proliferation in cultures being grown for transplantation or autotransplantation, as well as in diagnostics or screening procedures (e.g., in an assay drawn to sensitive hepatitis organisms). In some embodiments, tissues, cells or material treated in vitro or extra corporeally will, thereafter, be reintroduced into a subject (which need not be the source of origin of the tissue, cells or material). Compounds of the present invention can be employed in admixture with carriers, excipients and other drugs, and radiation therapy.

The compositions of this invention are generally administered to animals, including but not limited to mammals such as livestock, household pets, humans, cattle, cats, dogs, poultry, etc. Enteral and parenteral administration is contemplated within this invention.

For parenteral application, particularly suitable are injectable, sterile solutions, preferably oily or aqueous solutions, as well as suspensions, emulsions, or implants, including suppositories. Ampules are convenient unit dosages. Subcutaneous and i.v. administration are particularly contemplated.

For parenteral application, particularly suitable are tablets, dragees, liquids, drops, suppositories, or capsules. A syrup, elixir, or the like can be used wherein a sweetened vehicle is employed.

The pharmacologically active compositions of this invention can be processed in accordance with conventional methods of Galenic pharmacy to produce medicinal agents for administration to patients, e.g., mammals including humans.

The compositions of this invention can be employed in admixture with conventional excipients, i.e., pharmaceutically acceptable organic or inorganic carrier substances suitable for parenteral, enteral (e.g., oral or inhalation) or topical application which do not deleteriously react with the active compositions. Suitable pharmaceutically acceptable carriers include but are not limited to water, salt, sugar solutions, etc. The pharmaceutical preparations can be sterilized and if desired mixed with auxiliary agents, e.g. They can also be combined where desired with other active agents, including radiation or other antiviral or antineoplastic therapy. Particular reference is made to combined uses with antiviral drugs such as alpha interferon, ribavirin, amantadine, ganciclovir, acyclovir, zidovudine, foscarnet, dideoxycytosine, dideoxyinosine, rimantadine, stavudine, famciclovir, and trifluridine.

In some embodiments of the present invention, dosage forms include instructions for the use of such compositions.

For parenteral application, particularly suitable are injectable, sterile solutions, preferably suspensions. Ampules are convenient unit dosages.

Sustained or directed release compositions can be formulated, e.g., liposomes or those wherein the active component is protected with differentially degradable coatings, e.g., by microencapsulation, multiple coatings, etc. It is also possible to freeze-dry the new compositions and use the lyophilizates obtained, for example, for the preparation of products for injection.

Generally, the compositions of this invention are dispensed in unit dosage form comprising liposomal ATRA of from 15 to 300 or more mg/m² and particularly about 90 mg/m² ATRA, and from daily to about 5 out of 7 days to about 3 out of 7 days per week.

It will be appreciated that the actual preferred amounts of active compositions in a specific case will vary according to the specific compositions being utilized, the particular compositions formulated, the mode of application, and the particular situs and organism being treated. Dosages for a given subject can be determined using conventional considerations, e.g., by customary comparison of the differential activities of the subject compositions and of a known agent, e.g., by means of an appropriate, conventional pharmacological protocol.

1. A method of treating hepatitis comprising administering to a subject in need of such treatment a therapeutically effective amount of all-trans retinoic acid.

2. The method of claim 1 wherein said therapeutically effective amount of all-trans retinoic acid is at least about 10 mg at least about every other day.

3. The method of claim 1 wherein said all-trans retinoic acid liposomal all-trans retinoic acid.

4. The method of claim 3 wherein said therapeutically effective amount of liposomal all-trans retinoic acid is at least about 100 mg at least about every other day.

5. The method of claim 3 wherein said therapeutically effective amount of liposomal all-trans retinoic acid is at least about 65 mg/m² at least about every other day.

6. The method of claim 1 wherein said hepatitis is viral hepatitis

7. The method of claim 6 wherein the viral hepatitis is A, B, C, D, E, or G.

8. The method of claim 6 wherein the viral hepatitis is C.

9. The method of 3 wherein said amount is 10 mg/m².

10. The method of 9 wherein said amount is at least about 15 mg/m².

11. The method of 10 wherein said amount is at least about 50 mg/m².

12. A method of treating hepatitis comprising administering to a subject in need of such treatment a therapeutically effective amount of retinoid.

13. The method of claim 12 wherein said retinoid is in liposomal form.

14. The method of claim 12 wherein said therapeutically effective amount of retinoid is at least about 10 mg at least about every other day.

15. A method of treating enteroviral infection in an infected subject comprising administering to said subject a therapeutically effective amount of all-trans retinoic acid.

16. The method of claim 15 wherein said therapeutically effective amount of all-trans retinoic acid is at least about 10 mg at least about every other day.

17. The method of claim 15 wherein said all-trans retinoic acid liposomal all-trans retinoic acid.

18. The method of claim 17 wherein said therapeutically effective amount of liposomal all-trans retinoic acid is at least about 100 mg at least about every other day.

19. The method of claim 17 wherein said therapeutically effective amount of liposomal all-trans retinoic acid is at least about 65 mg/m² at least about every other day.

20. The method of claim 15 wherein said enteroviral infection is selected from the group consisting of (i) poliovirus, (ii) group A coxsackievirus, (iii) group B coxsackivirus, (iv) echovirus or (v) “newer” enterovirus.

23. The method of claim 15 wherein the enteroviral infection is selected from the group consisting of Echovirus 3, 4, 6, 7, 9 or 11, and Coxsackie A9, B2, B3, B5, or B9.

24. A method of treating enteroviral infection in an infected subject comprising administering to said subject a therapeutically effective amount of retinoid.

25. The method of claim 24 wherein said retinoid is in liposomal form.

26. The method of claim 24 wherein said therapeutically effective amount of retinoid is at least about 10 mg at least about every other day.

27. A method of treating chronic viral disease in a subject comprising administering to said subject a therapeutically effective amount of all-trans retinoic acid.

28. The method of claim 27 wherein said therapeutically effective amount of all-trans retinoic acid is at least about 10 mg at least about every other day.

29. The method of claim 27 wherein said all-trans retinoic acid liposomal all-trans retinoic acid.

30. The method of claim 29 wherein said therapeutically effective amount of liposomal all-trans retinoic acid is at least about 100 mg at least about every other day.

31. The method of claim 29 wherein said therapeutically effective amount of liposomal all-trans retinoic acid is at least about 65 mg/m² at least about every other day.

32. A method of treating chronic viral disease in a subject comprising administering to said subject a therapeutically effective amount of retinoid.

33. The method of claim 32 wherein said retinoid is in liposomal form.

34. The method of claim 32 wherein said therapeutically effective amount of retinoid is at least about 50 mg at least about every other day.
